(19) United States
(12) Patent Application Publication
(10) Pub. No.: US 2005/0170457 A1
(43) Pub. Date: Aug. 4, 2005

(54) Novel recombinant proteins with N-terminal free thiol

(76) Inventors: Chadler Pool, Phoenixville, PA (US); Juliane Mills, Wallingford, PA (US); Mark Cunningham, Kennett Square, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/021,516

(22) Filed: Dec. 23, 2004

Related U.S. Application Data
(60) Provisional application No. 60/533,617, filed on Dec. 31, 2003.

(51) Int. Cl. C12P 21/02; C07H 21/04; C07K 14/505; C12N 15/85

(52) U.S. Cl. 435/69.1; 435/455; 435/325; 530/350; 530/399; 536/23.5

(57) ABSTRACT

The present invention relates to novel modified proteins having N-terminal free thiols that can be produced by recombinant methods and are ready for further chemical derivatization. In particular, the invention relates to erythropoietin conjugate compounds having altered biochemical, physiochemical and pharmacokinetic properties. More particularly, one embodiment of the invention relates to erythropoietin conjugate compounds of the formula:
(M)ₙ-X-A-cys-EPO (I) where EPO is an erythropoeitin moiety selected from erythropoietin or an erythropoietin variant having at least one amino acid different from the wild-type human EPO, or any pharmaceutical acceptable derivatives thereof having biological properties of causing bone marrow cells to increase production of red blood cells; cys represents the amino acid cysteine and occurs at position −1 relative to the amino acid sequence of the erythropoietin moiety; A indicates the structure of the residual moiety used to chemically attach X to the thiol group of −1Cys; X is a water soluble polymer such as a polyalkylene glycol or other polymer; M is an organic molecule (including peptides and proteins) that increases the circulating half-life of the construct; and N is an integer from 0 to 15.

### CROSS REFERENCE TO RELATED APPLICATION

This application claims priority of U.S. provisional application Ser. No. 60/533,617, filed Dec. 31, 2003.

### FIELD OF THE INVENTION

The present invention relates to novel modified proteins that can be produced by recombinant methods and are ready for further chemical derivatization. In particular, the invention relates to erythropoietin conjugate compounds having altered biochemical, physiochemical and pharmacokinetic properties.

### BACKGROUND OF THE INVENTION

Erythropoietin (EPO) is a naturally formed glycoprotein which functions as a colony-stimulating factor and serves as the principal factor involved in the regulation of red blood cell synthesis. Erythropoietin acts by stimulating precursor cells in bone marrow causing them to divide and differentiate into mature red blood cells. This process is tightly controlled in the body such that the destruction or removal of red cells from the circulation is matched by the rate of new cell formation. Naturally occurring EPO is a glycoprotein produced in the kidney (Jacobs, et al. Nature 313 (6005), 806-810 (1985)). Thus, in addition to conditions of low or insufficient erythroblast production by precursors in marrow, any condition in which kidney function is compromised or destroyed, such as end-stage renal disease, represents an erythropoietin responsive condition.

Diverse cell types have been demonstrated to produce EPO and many cells in addition to erythroid progenitors express the EPO-Receptor, including capillary endothelial cells and in the brain. Astrocytes produce EPO in response to hypoxia (Masuda, S. et al. 1994 J Biol Chem 269: 19488-19493) and exogenous EPO could protect nearby neuronal cells from ischemic injury in an animal model (Sakanaka M., et al. 1998, Proc Natl Acad Sci USA 95:4635-4640) and thus EPO may have a role in protection and recovery from neurological damage or disease. More recently, erythropoietin has been found to protect retinal neurons from acute ischemia-reperfusion injury (Junk, et al. 2002, Proc. Nat. Acad. Sci. 99:10659-10664) and enhance neurological recovery from experimental spinal cord injury (Gorio et al., 2002, Proc. Nat. Acad. Sci. 99:9450-9455). Pathologic neural conditions, affecting neuronal or glial cells in the nervous system, can result from ischemia, apoptosis, necrosis, oxidative or free radical damage, and excitotoxicity. Neurological pathologies include, for example, cerebral and spinal ischemia, acute brain injury, spinal cord injury, retinal disease, and neurodegenerative diseases such as Alzheimer's disease, Parkinson's disease, Huntington's disease, and ALS. Therefore, exogenous EPO is now believed to be protective or preventive in some or all of these conditions.

In effect, EPO demonstrates endocrine (hormonal), autocrine and paracrine functions (activating or stimulating actions on self and neighboring cell types) in a variety of cells and tissue types (See Lappin, T. R. et al., 2002, Stem Cells 20:485-492 for a review) including myocardial tissue (Parsa, C. J. et al, 2003, J Clin Invest. 112(7): 999-1007) and gastrointestinal tissue (Fatouros, M. S., 2003, Eur J Surgery 165(10): 986-992). Thus, the potential therapeutic indications for administered EPO have expanded far beyond renal insufficiency and anemia.

Erythropoietin has been manufactured using recombinant DNA technology through the cloning of the EPO gene and expression in Chinese hamster ovary cells (Lin, U.S. Pat. No. 5,618,698). The recombinantly produced EPO has been available for some time as an effective therapeutic agent in the treatment of various forms of anemia, including anemia associated with chronic renal failure, zidovidine treated HIV infected patients, and cancer patients on myelosuppressive chemotherapy. The EPO glycoprotein is administered parenterally, either as an intravenous (IV) or subcutaneous (SC) injection in conventional buffered aqueous solutions which contain human serum albumin (HSA) as a carrier. Such formulations are marketed in the United States under the trade names EPOGEN® and PROCRIT®. These products contain erythropoietin in 1 ml single dose, preservative-free or 2 ml multidose preserved vials.

While these formulations have been proven to be highly successful, certain disadvantages are associated with the products. Presently, the period of bioactivity of protein therapeutics such as erythropoietin is limited by short plasma half-lives and the susceptibility to protease degradation. The short half-life of therapeutic proteins such as EPO, four hours, necessitates frequent administration for maximum clinical efficacy. This is disadvantageous for the treatment of chronic conditions and can result in poor patient compliance, and therefore less than optimal outcome. Accordingly, attempts have been made to increase the plasma half-life of EPO.

In recent years, non-antigenic water-soluble polymers, such as polyethylene glycol (PEG) have been used for the covalent modification of polypeptides of therapeutic and diagnostic importance. For example, covalent attachment of PEG to therapeutic polypeptides such as the interleukins (Knauf, M. J. et al., J. Biol. Chem. 1988, 263, 15,064; Tsutsumi, Y. et al., J. Controlled Release 1995, 33, 447), interferons (Kita, Y. et al., Drug Des Delivery 1990, 6, 157), catalase (Abuchowski, A. et al., J. Biol Chem. 1977, 252, 3, 582), superoxide dismutase (Beauchamp, C. O. et al., Anal Biochem. 1983, 131, 25), and adenosine deaminase (Chen, R. et al, Biochim, Biophys. Acta 1981, 660, 293), has been reported to extend their half-life in vivo, and/or reduce their immunogenicity and antigenicity.

Derivatized PEG compounds have been previously disclosed (U.S. Pat. No. 5,438,040). This approach to post-translational derivatization has also been applied to EPO. For example, WO 94/28024 discloses carbohydrate modified polymer conjugates with erythropoietin activity wherein the PEG is linked via an oxidized carbohydrate. U.S. Pat. No. 4,904,584 discloses polyalkylene oxide conjugation of lysine-depleted polypeptide variants, including EPO. WO 90/12874 describes the preparation of a monomethoxy-PEG-EPO (mPEG-EPO) in which the EPO contains a cysteine residue introduced by genetic engineering to which the specific PEG reagent is covalently attached. Other PEG-EPO compositions are disclosed in EP 605693, U.S. Pat. No. 6,077,939, WO 01/02017 and EP 539167.

Applicant's co-pending application U.S. Ser. No. 09/431,861 discloses the modification of antibodies and antibody fragments with PEG and demonstrates that PEG can increase circulating half-life in mice and primates. Derivatized PEG was used for modification of the Fab fragment of the antibody c7E3. Circulating half-life is increased in direct proportion to the molecular weight of the PEG. As the molecular weight of PEG increases, the ability of the compound to inhibit ADP-induced platelet aggregation in vitro is decreased, while the binding to purified GPIIb/IIIa, as measured by BIAcore, is unaffected. The addition of a fatty acid or a lipid to the PEG (PEG_{3.4K}-DSPE [disteroylphosphatidylethanolamine]) yielded a greater circulating half-life than did PEG_{5K}. While there is a decrease in the in vitro activity of c7E3 Fab′(PEG₅ₖ)₂ relative to c7E3 Fab, the activity of c7E3 Fab′-(PEG_{3.4k}-DSPE)₂ is equivalent to c7E3 Fab.

Applicant's other co-pending application U.S. Ser. No. 60/377,946 discloses methods for modifying EPO in which the EPO is covalently conjugated to a non-antigenic hydrophilic polymer covalently linked to an organic molecule that increases the circulating serum half-life of the composition more than what can be achieved by addition of a hydrophilic polymer alone. The methods include the step of reacting a protein or glycoprotein having erythropoietic activity with a substantially non-antigenic functionalized hydrophilic polymer having a linking group for attaching the polymer to the glycoprotein. Preparation methods include reacting EPO with an activated form of a polyalkylene oxide that will react with a functional group on EPO. This includes activated polyalkylene oxides such as active esters, hydrazide, hydrazine, semicarbazide, thiosemicarbazide maleimide or haloacetyl polyalkylene oxide.

An often limiting aspect of many methods of modifying proteins by conjugation to PEG (“PEGylation”) using purely chemical methods, is the indiscriminate and often incomplete reaction with amine groups which may occur on accessible lysine residues and/or the N-terminal amine of the protein. Other chemical methods require oxidation of the carbohydrate groups as part of the modification strategy likewise leading to incomplete or inconsistent reactions and undefined product compositions. Thus, considering the present options available, a method for modifying therapeutic proteins such as EPO in a mild, site-specific manner would be advantageous.

The modification or addition of motifs to a naturally occurring molecule carries multiple risks that are well known to those practicing the art to genetic engineering for the purposes of providing and manufacturing methods for therapeutic proteins. The most obvious of these effects is the loss or partial loss of biological activity. In other cases, the expression level from constructed expression vectors is unacceptably low from production cell lines. Another potential disadvantage is that coupling or fusion of a heterologous sequence even from a naturally occurring protein may create an antigenic epitope and cause unwanted immune reactions in the subject which ultimately limit the long term efficacy of the therapeutic protein. Furthermore, the modification of proteins using chemical methods that attack the most reactive functional group, lysine, also changes the isoelectric point of the protein and the pKa which may impact the structure and activity of the protein. Therefore, when the objective is to provide an active, safe and economically produced product, it is important to understand these limitations.

The introduction of cysteine residues has been shown to be an effective means of introducing a unique site on proteins for site-specific modifications (Kuan, Chien Tsun et al. Journal of Biological Chemistry 269, 7610-7616 (1994)). An N-terminal cysteine has particularly unique biochemical properties. Due to the close proximity of the alpha-amine and side chain thiol, N-terminal cysteine residues react with ester moieties to form stable amide bonds (Tam, James P. et al. Biopolymers 51, 311-332 (2000)). This allows for conjugation of peptides, proteins and other molecules to the N-terminus of a protein in a highly selective and stable manner. The presence of a free alpha-amine on cysteine also causes the local pH to be more alkaline, resulting in a higher reactivity of N-terminal thiols relative to thiols found on internal cysteines. As a consequence, another advantage is that conjugation reactions can be performed at lower pH resulting in less non-specific derivatization of the protein. A further advantage of the conversion of the thiol group of cysteine to a thioester, is that it does not result in a change in the isoelectric point or charge of that cysteine.

However, in secreted proteins, cysteine residues generally are present as the disulfide cystine, and contribute to the stabilization of the tertiary structure of the protein. Adding additional cysteine residues runs the risk of destabilizing the protein. For example, EPO contains four cysteine residues that are all involved in disulfide bridges. Thus, there is a possibility that introduction of a fifth cysteine residue at the N-terminus could interfere with proper folding and therefore receptor recognition.

The introduction of amino acid at a mature N-terminus provides an interesting challenge when modifying secreted proteins, namely the disruption of a signal sequence cleavage site. The vast majority of secreted proteins are translated with an additional region at the N-terminus where biosynthesis begins (called a signal or leader sequence), that targets the protein to the endoplasmic reticulum (ER). Signal sequences share certain features: they are usually from about 20 to 25 amino acids, are basic at the N-terminus, highly hydrophobic in the middle, and have small, uncharged residues preceding the site of cleavage by the signal peptidase. The hydrophobic region is essential for interaction with an ER receptor complex and facilitates translation and folding in an oxidizing environment. Upon secretion from membrane, the signal sequence is enzymatically cleaved at the functional mature N-terminal amino acid of the protein which becomes the only free alpha amine in the protein. The signal portion is retained and degraded inside the cell. Thus, the addition of an amino acid to the precursor protein sequence which will become the new N-terminal amino acid requires interposing an additional residue between the signal sequence and the normal mature N-terminus, thereby changing the native cleavage site with unknown impact on the efficiency of cleavage and secretion. The human EPO precursor polypeptide has a 27 amino acid signal sequence. Once in the ER compartment of the cell, the signal peptide is cleaved between glycine27 of the signal peptide and alanine28 of the mature EPO chain.

Genetic engineering methods can be used to add or change amino acids to a protein by adding or changing the nucleic acid coding sequence. Therefore, those skilled in the art will recognize the possibility of creating a novel therapeutic protein sequence that has a cysteine residue N-terminal of the naturally occurring N-terminal amino acid residue by manipulating the coding sequence or cDNA using standard techniques. To increase the probability that the N-terminus of an engineered protein would be cysteine, generally, the endogenous signal sequence must be replaced with one that is known to be efficient at targeting proteins to the ER, as well as produce a suitable cleavage site.

Heterologous signal sequences have successfully been used to engineer the mature N-terminus of proteins, for example, using alternative signal sequences for EPO expression in yeast (U.S. Pat. No. 4,775,622 and Elliott, S. et al. (1989) Gene 79, 167-180) and mammalian cells (Kim, Chang H. et al. (1997) Gene 199, 293-301). However, there are no reports of a heterologous signal sequence being used to secrete an N-terminally engineered form of a therapeutic protein.

### SUMMARY OF THE INVENTION

The invention provides biologically active polypeptide conjugate compositions wherein the polynucletide sequence coding for the polypeptide is modified to produce a conjugation partner peptide have an N-terminal cysteine and which partner is covalently and site specifically conjugated to a non-antigenic hydrophilic polymer that can also be covalently linked to an organic molecule either of which modification increases the circulating serum half-life of the composition.

More particularly, one embodiment of the invention thus relates to EPO derivatives described by the formula
(M)ₙ-X-A-cys-EPO (I)
where EPO is an erythropoeitin moiety selected from erythropoietin or an erythropoietin variant having at least one amino acid different from the wild-type human EPO, or any pharmaceutical acceptable derivatives thereof having biological properties of causing bone marrow cells to increase production of red blood cells; cys represents the amino acid cysteine and occurs at position −1 relative to the amino acid sequence of the erythropoietin moiety; A indicates the structure of the residual moiety used to chemically attach X to the thiol group of −1Cys; X is a water soluble polymer such as a polyalkylene glycol or other polymer; M is an organic molecule (including peptides and proteins) that increases the circulating half-life of the construct; and N is an integer from 0 to 15. Other molecules may be included between A and X or between X and M to provide the proper functionality for coupling or valency. The organic molecule, M, is optional. X is preferably a polyalkylene oxide such as polyethylene glycol and is also optional.

The invention also provides methods of treating anemia or other conditions associated with reduced endogenous erythropoietin or erythropoiesis or conditions under which an increase in red cells is desired. The methods of the invention also include the use of the compositions of the invention to treat conditions not directly linked to erythropoietic deficiency but that may be related to the anti-apoptotic effects of EPO associated with maintenance or enhancement of muscle, mucosal tissue, gonadal function and cognitive function. The methods of the invention further include the use of the compositions of the invention to protect, maintain, or treat neurological tissue or other tissues from ischemic, chemical or mechanical damage. In this aspect of the invention, treatment includes administering an effective amount of the conjugates described herein to mammals requiring such therapy. As a result of the present invention, conjugates having substantially prolonged erythropoietic activity in vivo and methods for producing said conjugate are provided.

Advantages of the techniques disclosed herein are a substantially defined end-product composition achieved through expression of an EPO variant containing an N-terminal cysteine residue and increased half-life of EPO.

### BRIEF DESCRIPTION OF THE DRAWINGS

FIG. 1 shows the amino acid sequence of a re-engineered precursor erythropoietin molecule based on the 166 amino acid human form and with the −1cys residue shown in a box and the signal sequence shown in bold.

FIG. 2 shows a stained SDS PAGE analysis of purified cys-EPO and N-terminal sequence of purified cys-EPO as determined by chemical methods, (N) denotes a placeholder.

FIG. 3 is a graph showing the results of a UT-7 cell proliferation assay comparing cys-EPO with EPO.

FIG. 4 shows a 4-12% SDS-PAGE gel of samples as indicated in lanes: (1) molecular weight markers; (2) EPO+0 mM MEA; (3) EPO+0 mM MEA+maleimide-PEG; (4) EPO+15 mM MEA; (5) EPO+15 mM MEA+maleimide-PEG; (6) EPO+20 mM MEA; (7) EPO+20 mM MEA+maleimide-PEG; (8) EPO+25 mM MEA; (9) EPO+25 mM MEA+maleimide-PEG; (10) EPO standard.

FIG. 5 shows a 4-12% SDS-PAGE gel of samples as indicated in lanes: (1) molecular weight markers; (2) Cys-EPO+0 mM MEA; (3) Cys-EPO+0 mM MBA+maleimide-PEG; (4) Cys-EOP+15 mM MEA; (5) Cys-EPO+15 mM MEA+maleimide-PEG; (6) Cys-EPO+20 mM MEA; (7) Cys-EPO+20 mM MEA+maleimide-PEG; (8) Cys-EPO+25 mM MEA; (9) Cys-EPO+25 mM MEA+maleimide-PEG; (10) Cys-EPO standard. Note that the bands corresponding to Cys-EPO conjugated to PEG are indicated by the white arrow.

FIG. 6 is a SELDI mass spectra of EPO, EPO+0 mM MEA+maleimide-PEG, and EPO+25 mM MEA+maleimide-PEG. The peaks around 28,000 correspond to unmodified EPO.

FIG. 7 is a SELDI mass spectra of Cys-EPO, Cys-EPO+0 mM MEA+maleimide-PEG, and Cys-EPO+25 mM MEA+maleimide-PEG. The peaks around 28,000 correspond to unmodified cys-EPO with the presence of peaks corresponding to the addition of a 5,960 MW PEG.

### DETAILED DESCRIPTION

The proteins of the invention are N-cys variants of therapeutic proteins and have an N-terminal free thiol or “NTFT” allowing for specific and stable chemical modification to be affected. Very few naturally occurring or recombinantly made proteins are processed in such a way that a cysteine residue is the mature N-terminus of the molecule. The method of the invention uses a heterologous signal sequence to ‘force’ the presentation of an added cysteine at the N-terminus of a mature, naturally occurring human protein or engineered protein of therapeutic value. The use of a heterologous signal sequence avoids the potential for inaccurate or mis-processing of the desired N-terminal cysteine residue that can occur if the endogenous leader cannot accommodate such a change. Poor processing or inaccurate processing results in a) poor cleavage efficiency of the signal sequence leading to a poor expression rate or b) inappropriate cleavage at the N-terminus either not leaving the cysteine at all or leaving it in the +2, +3 or +n position. In the latter case, the specificity and ease of chemical modification of the protein will not be as optimal as when the cysteine is at the +1 position in the mature protein.

Precursor proteins comprising a processing or “signal” peptide targeting specific proteins for extracellular secretion were noted as early a 1972 for immunoglobulin proteins and more recently the substructure of these sequences as well as the associated processing steps and enzymes have been studied in more detail (See Dalbey, et al. (1997) Protein Sci. 6:1129-1138, for an overview).

One particularly preferred signal sequence is the human growth hormone leader sequence (SEQ ID NO: 2) however, in theory, there are many mammalian heterologous leader sequences that could work efficiently and accurately in generating the required N-terminus. Other mammalian precursor polypeptides which comprise signal peptides that yield mature N-terminal cysteine proteins are those associated with the interferon alpha gene family. In silico prediction algorithms have been developed, such as SigCleave which is a weighted matrix method (EMBOSS) and SigPfam which is based on a hidden Markov model (HMM) to predict the probability that a protein comprises as signal peptide and the mostly likely cleavage sites. Various signal sequences from human precursor proteins are shown in Table 1 along with the predicted cleavage site when coupled to N-Cys-EPO as a the desired mature protein using the SignalP Version 3.0 (www.cbs.dtu.dklservices/SignalP/; Bendtsen et al. J. Mol. Biol., 340:783-795, 2004). The SignalP v3.0 provides both a neural network (NN) trained on eukaryotic proteins from the Swiss-Prot database of experimentally verified cleavage sites and a HMM. Based on these predictions (Table 1) shows that the native EPO leader sequence is predicted to be unsuitable for as a leader for N-Cys-EPO while the hGH leader and some but not all of the interferon (IFN) protein signal peptides are predicted to yield a protein with and N-terminal cys.

TABLE 1

| | | | NCBI | SignalP 3.0 | SignalP 3.0 |
|---|---|---|---|---|---|
| | | Protein | Accession | (NN) | (HMM) |
| Signal Sequence | Length | Name | No. | Most likely | Probability |
| MGVHECPAWL WLLLSLLSLP | 27 | EPO | P01588 | 28 and 29: | 0.476 between |
| LGLPVLG (SEQ ID NO: 1, | | | | LGC-AP | 27 and 28 |
| 1-27) | | | | | |
| MATGSRTSLL LAFGLLCLPW | 26 | Growth | NP_000506 | 26 and 27: | 0.490 between |
| LQEGSA (SEQ ID NO: 2) | | Hormone, | | GSA-CA | 27 and 28 |
| | | isoforms | | | |
| MAWVWTLLFL MAAAQSIQA | 19 | Antibody | | 19 and 20: | 0.777 between |
| (SEQ ID NO: 3) | | HC | | IQA-CA | 19 and 20 |
| MGIKMETHSQ VFVYMLLWLS | 25 | Antibody | | 25 and 26: | 0.878 between |
| GSVEG (SEQ ID NO: 4) | | LC | | VEG-CA | 25 and 26 |
| MASPFALLMV LVVLSCKSSC | 23 | IFNalpha1 | NP_076918 | 23 and 24: | 0.324 between |
| SLG (SEQ ID NO: 5) | | | | SLG-CA | 23 and 24 |
| MALTFALLVA LLVLSCKSSC | 23 | IFNalpha2 | NP_000596 | SVG-CA | 0.333 between |
| SVG (SEQ ID NO: 6) | | | | | 20 and 21 |
| MALSFSLLMA VLVLSYKSIC | 23 | IFNalpha4, | NP_066546 | 23 and 24: | 0.222 between |
| SLG (SEQ ID NO: 7) | | 10, & 17 | | SLG-CA | 23 and 24 |
| MALPFVLLMA LVVLNCKSIC | 23 | IFNalpha5 | NP_002160 | 16 and 17: | 0.205 between |
| SLG (SEQ ID NO: 8) | | | | LNC-KS | 22 and 23 |
| MALPFALLMA LVVLSCKSSC | 23 | IFNalpha6 | NP_066282 | 24 and 25: | 0.375 between |
| SLD (SEQ ID NO: 9) | | | | LDC-AP | 21 and 22 |
| MALSFSLLMA VLVLSYKSIC | 23 | IFNalpha10 | NP_002162 | Same as | |
| SLG (SEQ ID NO: 7) | | | | IFNalpha 4 | |
| MASPFALLMA LVVLSCKSSC | 23 | IFNalpha13 | NP_008831 | 23 and 24: | 0.324 between |
| SLG (SEQ ID NO: 10) | | | | SLG-CA | pos. 23 and 24 |
| MALPFALMMA LVVLSCKSSC | 23 | IFNalpha14 | NP_002163 | 23 and 24: | 0.320 between |
| SLG (SEQ ID NO: 11) | | | | SLG-CA | pos. 23 and 24 |
| MALSFSLLMA VLVLSYKSIC | 23 | IFNalpha17 | NP_067091 | Same as | |
| SLG (SEQ ID NO: 7) | | | | IFNalpha 4 | |
| MAFVLSLLMA LVLVSYGPGG | 23 | IFNdelta1 | P37290 | 23 and 24: | 0.815 between |
| SLG (SEQ ID NO: 12) | | | | SLG-CA | pos. 23 and 24 |
| MALLFPLLAA LVMTSYSPVG | 23 | IFNomega1 | NP_002168 | 23 and 24: | 0.649 between |
| SLG (SEQ ID NO: 13) | | | | SLG-CA | pos. 23 and 24 |

A variety of mammalian expression vectors have been used successfully to express recombinant proteins. An exemplary composition produced by the method of the invention comprises a simple expression vector utilizing a strong viral promoter, a consensus Kozak sequence, the DNA coding for the precursor of the protein of interest (hHG signal peptide:EPO), a hexaHis tag, a stop codon and a polyadenylation signal derived from, for example, the SV40 (simian virus) polyadenylation signal or the bovine growth hormone polyadenylation signal.

Once an expression vector containing the composition of the invention has been constructed, the novel protein is expressed using conventional methods of transfecting a host cell. Transient transfection or stable transfection methods can be used and any host cell (preferred mammalian), capable of processing mammalian signal sequences could be used. Examples of useful host cell lines are VERO and HeLa cells, Chinese Hamster Ovary (CHO) cell lines, W138, 293, BHK, COS-7 and MDCK cell lines. Methods of recovery and purification of proteins from cell cultures are well known to those skilled in the art and include the addition of addition coding regions for amino acid motifs useful for called purification “tags” such as hexa-histidine or FLAG.

When expressed by prokaryotes the polypeptides typically contain an N-terminal methionine or a formyl methionine and are not glycosylated thus are not preferred. Those skilled in the art will recognize, however, that the present invention is not limited to the use of the aforementioned mammalian signal peptide containing vector and mammalian host cells particularly when it is not the objective to produce a glycosylated protein. Bacterial systems for the expression and secretion of proteins are known and used. For example, the Staphylococcus aureus nuclease signal peptide coding sequence has been used in a construct for the production of proinsulin and Bacillus, see for example EP0176320A1. Various secretory signal peptide sequences can be useful in Bacillus and, subject to manipulation, can produce the N-terminal cysteine polypeptides of the invention. Such secretion coding sequences include, but are not limited to, the alpha-amylase signal peptide sequence of B amyloliquifaciens, the 0-lactamase Type I signal peptide sequence of B cereus, the B. subtilis levansucrase signal peptide sequence) and the B amyloliquefaciens subtilisin signal peptide sequence. The above secretory coding sequences can be appropriately ligated between the transcriptional and translational activating sequence of the vector and the sequence that codes for a functional N-terminal cysteine polypeptide.

EPO

EPO is primarily produced in the kidneys and functions through binding to receptor dimers on precursor cells leading to differentiation to erythrocytes and subsequent proliferation (Livnah, O. et al. Science 1999, 283, 987-990). EPO binds to the receptors through two binding surfaces, one of which has a higher affinity for the receptor than the other. The crystal structure of EPO has been solved (Syed, et al. Nature 395 (6701), 511-516 (1998); Cheetham, J. C. et al. Human Erythropoietin, NMR minimized average structure. 8-Sep-1998. Protein data base ID 1BUY). The crystal structure of EPO binding to its receptors has also been described (see Stroud, R. M. and Reid, S. W., Erythropoietin complexed with extracellular domains of erythropoietin receptor. Protein data base ID 1CN4).

The erythropoietin gene has 5 exons that code for a 193-amino acid pro-polypeptide (SEQ ID NO: 1). A 27-amino acid leader sequence is cleaved off the amino terminus of the pro-polypeptide, yielding the functional 166-amino acid polypeptide. However, recombinant human erythropoietin expressed in Chinese hamster ovary cells contains only 165 amino acids, having lost arg166. The mechanism for this is undefined, and whether erythropoietin circulating in the plasma also lacks arg166 or further C-terminal truncation is not known. Both the nucleotide and amino acid sequences of erythropoietin are highly conserved among mammals.

The starting material for modification to a bioactive form of EPO of the invention is preferably, human erythropoietin (SEQ ID NO. 1) or des-166Arg SEQ ID NO: 1, or other variants known to possess biological activity, or other derivatives thereof having the biological properties of causing bone marrow cells to increase production of reticulocytes and red blood cells. The EPO glycoprotein may be obtained from natural sources or produced recombinantly using known procedures as disclosed in U.S. Pat. Nos. 4,703,008; 5,441,868; 5,547,933; 5,618,698 and 5,621,080 hereby incorporated by reference. In the wild-type human EPO, Asn residues at position 24, 38, and 83 represent the three naturally occurring N-linked glycosylation sites. Glycosylation at these three positions and one O-linked site (Ser123) account for about 40% of the weight of both natural and recombinant EPO produced in mammalian cell cultures. Genetically modified variants have been created with more, fewer, or different glycosylation sites. Nonglycosylated forms, hypoglycosylated or hyperglycosylated forms of erythropoietin protein with the desired biological activity may also be used in the compositions of the invention. Nonglycosylated proteins are produced by prokaryotic organisms, therefore the use of codon adapted nucleic acid sequences for mammalian proteins in expression systems using prokaryotic cells, such as E. coli, results in the ability to produce nonglycosylated protein product. Such a method is taught in WO00/32772. Other variants with altered glycosylation patterns made by amino acid exchange of any of the Asn residues at position 24, 38 or 83 with biological activity have also been described (Yamaguchi, K., et al., 1991, J. Biol. Chem. 266: 20434-20439).

Methods of producing hyperglycosylated EPO are taught in WO0249673 and EP640619. Additional N-linked carbohydrate chains can be added to the rHuEPO molecule. In mammalian cells, N-linked carbohydrate is attached to the polypeptide backbone at a consensus sequence for carbohydrate addition (Asn-X-Ser/Thr) where X is an amino acid except Pro or Asp. This process occurs in the cellular endoplasmic reticulum and is catalyzed by a membrane-bound oligosaccharide transferring enzyme. Knowledge of the recognition consensus consequence has been exploited by genetic engineers to introduce new carbohydrate attachment sites into a polypeptide backbone by making the requisite changes in the sequence of the DNA to be cloned. Of course, the consensus sequences must be added intelligently at positions that are compatible with carbohydrate addition, for example, positions that do not interfere with receptor binding, or compromise the folding, conformation, or stability of the molecule. The erythropoietin analog, NESP, was generated by combining the carbohydrate addition sites of 2 successfully glycosylated 4-chain analogues into one molecule. The amino acid sequence of NESP differs from that of human erythropoietin at 5 positions (Ala30Asn, His32Thr, Pro87Val, Trp88Asn, and Pro90Thr) allowing for additional oligosaccharide attachment at asparagine residues at positions 30 and 88 (Elliott et al, Blood 96:82a (2000)). The hyperglycosylated variants disclosed in patent application publication WO0181405 are those with three additional N-linked glycosylation sites at: 30, 53, and 88; 30, 55, and 114; or 30, 88, and 114.

Pegylated EPO variants with altered glycosylation have also been described, U.S. Pat. No. 6,583,272 and U.S. Pat. No. 6,340,742, wherein the added glycosylation consensus sequences are principally at positions 30, 57, 59, 67, 88, 89, 136, and 138.

Methods of Re-Engineering Proteins

The polypeptide variants, or functional fragments thereof, of the invention can be generated using any of several methods known in the art. Oligonucleotide-directed mutagenesis is a well-known and efficient procedure for systematically introducing mutations, independent of their phenotype and is, therefore, suited for directed evolution approaches to protein engineering. The methodology is flexible, permitting precise mutations to be introduced without the use of restriction enzymes, and is relatively inexpensive. Recombinant and enzymatic synthesis, including polymerase chain reaction and other amplification methodologies can be found described in, for example, Sambrook et al., Molecular Cloning: A Laboratory Manual, Third Ed., Cold Spring Harbor Laboratory, New York (2001) and in Ausubel et al., Current Protocols in Molecular Biology, John Wiley and Sons, Baltimore, Md. (1999).

Methods for efficient synthesis and expression of mutated polypeptides synthesized using oligonucleotide-directed mutagenesis can be performed, and is well known in the art as described by Adelman et al., (1983) DNA, 2:183 and Kunkel, Proc. Natl. Acad. Sci. USA, 82:488-492 (1985) which are incorporated herein by reference.

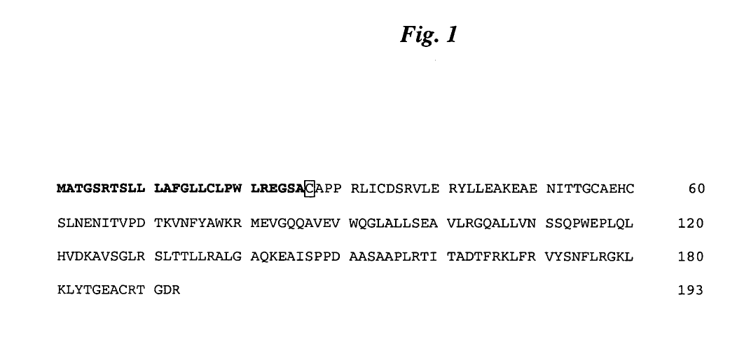

Also, for example, single or multiple amino acids mutations can be generated using oligonucleotides that code for the mutated amino acid(s) such as utilized in PCR based site-directed mutagenesis (for example, QuikChange TM, Stratagene). Site-directed mutagenesis of cDNA encoding wild-type or parent protein can also be achieved using the techniques described by Higuchi et al., Nucleic, Acids Res. 16:7351-7367 (1988), which is incorporated herein by reference. In general, this procedure calls for the use of two sets of primers: a first pair which flanks the entire cDNA of protein to be mutated and which therefore will produce a full length copy of the cDNA upon PCR amplification, and a second pair which are complementary to one another and which contain the desired mutation. These primers initially produce two sets of products, one having the mutation introduced near the 3′-end, and the other having the mutation introduced near the 5′-end. Because these two products are complementary to one another as well as to the PCR primers, however, the two products can form an overlapping duplex which is extended in both directions. Thus, PCR amplification of cDNA in the presence of two primer sets can be used to generate a full-length cDNA (SEQ ID NO: 14) coding for the desired construct as shown in FIG. 1.

Synthetic or, at a minimum, cell free methods of manufacturing a gene or gene fragment is also well within the known art. Methods for synthesizing large nucleic acid polymers by sequential annealing of oligonucleotides can be found described in, for example, in PCT application No. WO 99/14318 and in U.S. Pat. No. 6,521,427, both to Evans. Methods of synthesizing altered genes or complete coding sequences using in vitro methods have previously been applied to human EPO: see, for example; U.S. Pat. No. 6,159,687 and U.S. Pat. No. 6,537,746. Methods of directing mutations in vivo to produce EPO and other proteins with altered properties is taught in EP0843725 and U.S. Pat. No. 6,444,441.

The method of the invention for adding cysteine at the N-terminus of an active protein such as EPO or EPO variant polypeptide can be practiced using any of the variant or mutant forms of the protein having the desired biological activity. The method may be practiced on glycoproteins, especially of EPO, expressed using eukaryotic cell systems or may be practiced on asialated or completely aglycosylated proteins or EPO.

Water Soluble Polymers

A particularly preferred water-soluble polymer is one of the several species of PEG. PEG consists of a basic carbon unit, HO—(CH2)2-OH, and is sold in various forms under the names: Polyethylene glycol (various molecular weights); PEG 200; PEG (various molecular weights); poly ethylene oxide; Carbowax; alpha-hydro-omega-hydroxypoly(oxy-1,2-ethanediyl); ethoxylated 1,2-ethanediol; polyoxyethylene ether; Emkapol 200; Gafanol e 200; Pluriol e 200; Polydiol 200; Polyox WSR-301; Macrogol; and polyoxyethleneln. In those aspects of the invention in which PEG-based polymers are used, it is preferred that they have average molecular weights between about 200 and about 100,000 Daltons, and preferably between about 2,000 and about 20,000 Daltons. A molecular weight of 2,000 to 12,000 Daltons is most preferred.

Alternative water soluble polymeric substances include materials such as dextrans, polyvinyl pyrrolidones, polysaccharides, starches, polyvinyl alcohols, polyacrylamides or other similar non-immunogenic polymers. Those of ordinary skill in the art realize that the foregoing is merely illustrative and unintended to restrict the type of non-antigenic polymers suitable for use herein.

Organic Molecule Imparting Extended Pharmacokinetic Half-Life in vivo

The organic moieties that can be attached to the hydrophilic polymer to increase the half-life include fatty acids, dicarboxylic acids, monoesters or monoamides of dicarboxylic acids, lipids containing saturated fatty acids, lipids containing unsaturated fatty acids, lipids containing mixtures of saturated and unsaturated fatty acids, simple carbohydrates, complex carbohydrates, carbocycles (such as steroids), heterocycles (such as alkaloids), amino acid chains, proteins, enzymes, enzyme cofactors, or vitamins.

In one embodiment, the hydrophilic polymeric group is substituted with one to about six alkyl, fatty acid, fatty acid ester, lipid or phospholipid groups (as described herein, e.g., Formula I). Preferably, the substituted hydrophilic polymeric group is a linear or branched PEG. Preferably, the substituted hydrophilic polymeric group is a linear PEG (e.g., PEG diamine) that is terminally substituted with a fatty acid, fatty acid ester, lipid or phospholipid group or a hydrocarbon. Hydrophilic polymers that are substituted with an alkyl, fatty acid, fatty acid ester, lipid or phospholipid groups group can be prepared using suitable methods. For example, a modifying agent can be prepared by reacting monoprotected PEG diamine with an activated fatty acid (e.g., palmitoyl chloride). The resulting product can be used to produce a modified EPO that comprises a PEG that is substituted with a fatty acid group. A variety of other suitable synthetic schemes can be used. For example, an amine containing polymer can be coupled to a fatty acid or fatty acid ester as described herein, and an activated carboxylate (e.g. activated with N,N′-carbonyl diimidazole) on a fatty acid or fatty acid ester can be coupled to an hydroxyl group on a polymer. In this way, a multitude of suitable linear and branched chain multimeric structures having the desired properties can be constructed and finally linked or modified to contain a primary amine which will act as the transglutaminase amine donor.

Fatty acids and fatty acid esters suitable for use in the present invention can be saturated or can contain one or more unsaturated units. In a preferred embodiment, the fatty acids and fatty acid esters comprise from about six to about forty carbon atoms or about eight to about forty carbon atoms. Fatty acids which are suitable for modifying EPO in the method of the invention include, for example, n-dodecanoate (C12, laurate), n-tetradecanoate (C14, myristate), n-hexadecanoate (C16, palmitate), n-octadecanoate (C18, stearate), n-eicosanoate (C20, arachidate), n-docosanoate (C22, behenate), n-triacontanoate (C30), n-tetracontanoate (C40), cis- Δ9-octadecanoate (C18, oleate), all cis-Δ5,8,11,14-eicosatetraenoate (C20, arachidonate), octanedioic acid, tetradecanedioic acid, octadecandeioic acid, docosanedioic acid, and the like. Suitable fatty acid esters include monoesters of dicarboxylic acids which comprise a linear or branched lower alkyl group. The lower alkyl group can comprise from one to about twelve, preferably one to about six, carbon atoms. Suitable fatty acid esters for modifying proteins of the invention include, for example, methyl octadecanoate, ethyl octadecanoate, propyl octadecanoate, butyl dodecanoate, sec-butyl dodecanoate, tert-butyl dodecanoate, neopentyl tetradecanoate, hexyl tetradecanoate, methyl cis-Δ9-octadecanoate, and the like.

Preparation of the Substrate for Transfer to an N-Terminal Cys Polypeptide

Compositions comprising two or three components or more linked to an electrophile can function as a suitable conjugation substrate in the processes of the present invention.

The preparation of substrates is preferably performed stepwise and in the final step will result in a single thiol reactive compound. Disulfide linkages and thioester linkages, which are cleaved by reducing agents such as DTT, and thioether linkages, which are not cleavable under reducing conditions can be used in the compositions of the invention.

Formation of disulfide linkages is achieved using a thiol-containing substrate or an activated disulfide, namely PEG-orthopyridyl-disulfide (C. Woghiren, B. Sharma, S. Stein, Protected thiol-polyethylene glycol: a new activated polymer for reversible protein modification, Bioconjugate Chem 4 (1993) 314). Thioether linkages are conveniently formed using a maleimide activated substrate or with PEG-iodoacetamide. A relatively new reagent, based on thiol addition to PEG-vinylsulfone double bond has also been demonstrated (M. Morpurgo, O. Schiavon, P. Caliceti, F. M. Veronese, Covalent modification of mushroom tyrosinase with different amphiphic polymers for pharmaceutical and biocatalysis applications, Appl Biochem Biotechnol 56 (1996) 59-72). Other methods of conjugating organic molecules to polymers are well known and include the use of agents which react with thiols, for example, acrylolyl, pyridyl disulfides, 5-thiol-2-nitrobenzoic acid thiol (TNB-thiol), and the like.

A reactive group can be bonded directly to the hydrophilic polymer, conjugate complex or through a linker moiety, for example a C1-C12 hydrocarbyl group. As used herein, “hydrocarbyl group” refers to a hydrocarbon chain wherein one or more carbon atoms are optionally replaced by a heteroatom such as oxygen, nitrogen or sulfur. Suitable linker moieties include, for example, tetraethylene glycol, —(CH2)₃-,—NH—(CH2)₆-NH—, —(CH₂)₂—NH— and —CH₂—O—CH₂—CH₂—O—CH₂—CH₂—O—CH—NH—.

Linkage of water soluble polymer X and lipophilic agent M may be performed prior to the conjugation of the final substrate to the N-terminal cysteine of the protein and use any chemical or enzymatic method known in the art. Thus, if for example, amine-reactive groups include electrophilic groups such as tosylate, mesylate, halo (chloro, bromo, iodo), N-hydroxysuccinimidyl esters (NHS), substituted phenyl esters, acyl halides and the like are to be used to couple water soluble polymer and organic molecules, the primary amine in most cases must be protected. Other methods of conjugating organic molecules to polymers are well known and include the use of agents which can react with thiols, for example, maleimide, iodoacetyl, acrylolyl, pyridyl disulfides, 5-thiol-2-nitrobenzoic acid thiol (TNB-thiol), and the like. An aldehyde or ketone functional group can be coupled to amine-or hydrazide-containing molecules and an azide group can react with a trivalent phosphorous group to form phosphoramidate or phosphorimide linkages. Suitable methods to introduce such thiol reactive groups into molecules are known in the art (see for example, Hermanson, G. T., Bioconjugate Techniques, Academic Press: San Diego, Calif. (1996). A reactive group can be bonded directly to the hydrophilic polymer, conjugate complex or through a linker moiety, for example a C1-C12 hydrocarbyl group. As used herein, “hydrocarbyl group” refers to a hydrocarbon chain wherein one or more carbon atoms are optionally replaced by a heteroatom such as oxygen, nitrogen or sulfur. Suitable linker moieties as used between the cysteine and the substrate may also be used between components of the substrate composition and include, for example, tetraethylene glycol, —(CH2)₃-,—NH—(CH2)₆-NH—, —(CH₂)₂—NH— and —CH₂—O—CH₂—CH₂—O—CH₂—CH₂—O—CH—NH—.

Modifying agents which comprise a linker moiety can be produced, for example, by reacting a mono-Boc-alkyldiamine (e.g. mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoracetic acid (TFA) to expose a primary amine which can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference).

Conjugation of Substrate to the N-Terminal Cys of the Polypeptide

Once the polymeric moiety is selected for conjugation to the N-terminal cys of the polypeptide and is synthetically prepared, if not already activated, the polymeric substrate must be activated with a thiol reactive group at the position desired to be linked to the N-terminus of the polypeptide.

Sulfones

Synthetic routes used to prepare active sulfones of poly(ethylene glycol) and related polymers are taught in U.S. Pat. No. 5,446,090 (Shearwater) which is incorporated in its entirety herein by reference. According to the teaching in the patent, the process comprises at least four steps in which sulfur is bound to a polymer molecule and then converted through a series of reactions to an active sulfone functional group. A further reaction (5) is the conversion of the haloalkylsulfone to vinyl sulfone under basic conditions. Particularly preferred reagents for use in each step are shown herein below but other reagents can also be employed:
(1) PEG-OH+CH₃SO₂Cl→PEG-OSO₂CH₃
(2) PEG-OSO₂CH₃+HSCH₂CH₂OH→PEG-SCH₂CH₂OH
(3) PEG-SCH₂CH₂OH+H₂O₂→PEG-SO₂CH₂CH₂OH
(4) PEG-SO₂CH₂CH₂OH+SOCl₂→PEG-SO₂CH₂CH₂Cl
(5) PEG-SO₂—CH₂CH₂Cl+NaOH→PEG-SO₂—CH=CH═HCl

Step 1 is the “activation” of an available hydroxyl on the hydrophilic polymer. The hydroxyl moiety will be activated typically by one of two routes, hydroxyl esterification or substitution, although other methods are available as should be apparent to the skilled artisan. Hydroxyl esterification is accomplished by reacting an acid or an acid derivative such as an acid halide with the PEG to form a reactive ester. Typical esters are the sulfonate, carboxylate, and phosphate esters. Sulfonyl acid halides that are suitable for use in practicing the invention include methanesulfonyl chloride and p-toluenesulfonyl chloride.

In substitution, the —OH group of the hydrophilic polymer is substituted by a more reactive moiety, typically a halide. For example, thionyl chloride, represented structurally as SOCl₂, can be reacted with PEG to form a more reactive chlorine substituted PEG.

Step 2 is to link sulfur directly to a carbon atom in the polymer and form an ethyl sulfone or ethyl sulfone derivative having similar reactive properties. The 2 carbon “Ethyl” moiety is required so that the second carbon atom in the chain away from the sulfone group provides a reactive site for linkages of thiol moieties with the sulfone.

Step 3 involves limited oxidation of sulfur that is attached to the carbon to the sulfone group, —SO₂. There are many such oxidizing agents, including hydrogen peroxide and sodium perborate. A catalyst, such as tungstic acid, can be useful.

In Step 4, the hydroxyl moiety added in the second step is converted to a more reactive form, either through activation of the hydroxyl group or through substitution of the hydroxyl group with a more reactive group, similar to the first step in the reaction sequence.

The resulting polymeric activated ethyl sulfone is stable, isolatable, and suitable for thiol-selective coupling reactions. For example, PEG chloroethyl sulfone is stable in water at a pH of about 7 or less, but nevertheless can be used to advantage for thiol-selective coupling reactions at conditions of basic pH up to at least about pH 9. PEG vinyl sulfone is also stable and isolatable and can form thiol-selective, hydrolytically stable linkages.

Step 5, can be added to the synthesis, to convert the activated ethyl sulfone to a vinyl sulfone or one of its active derivatives for use in thiol-selective coupling reactions. Polymer vinyl sulfone reacts more rapidly with thiols than its ethyl sulfone counterpart and is stable against hydrolysis in water of pH less than about 11 for at least several days.

The reaction is expected to produce a product in which the ethyl or vinyl carbons remain as part of the final conjugate. U.S. Pat. No. 5,446,090 and the teachings therein provide active PEG sulfones of any molecular weight and can be linear or branched may be substituted or unsubstituted. The stability of the sulfone moiety against hydrolysis makes it particularly useful for bifunctional or heterobifunctional applications.

Polymer vinyl sulfone and its precursors and derivatives can be used for attachment directly to surfaces and molecules having a thiol moiety. However, more typically a heterobifunctional hydrophilic polymers such as a PEG having both an ethyl sulfone moiety at one position, typically near the end of the polymer, and a different functional moiety on the opposite end. A heterobifunctional PEG having a sulfone moiety on one end and an amine specific moiety on the other end could be attached to both cysteine and lysine fractions of, for example, the same or different proteins. Alternatively, a heterobifunctionalized molecule could be used to incorporate a second organic moiety as described herein, in so far as a stable amine linkage can be formed prior to reaction of the unreacted sulfone moiety.

Other active groups for heterobifunctional activated PEGs can be selected from among a wide variety of compounds. For biological and biotechnical applications, the substituents would typically be selected from reactive moieties typically used in PEG chemistry to activate PEG such as the aldehydes, trifluoroethylsulfonate, which is also sometimes called tresylate, n-hydroxylsuccinirnide ester, cyanuric chloride, cyanuricfluoride, acyl azide, succinate, the p-diazo benzyl group, the 3-(p-diazophenyloxy)-2-hydroxy propyloxy group, maleimide, and others.

The attachment of peptides, proteins, PEGs and other polymers to an N-terminal cysteine-containing EPO can also be achieved through ester chemistries. For the case of peptides and proteins, both can be synthesized or expressed to contain ester moieties (preferentially thioester) at their C-termini. Under mild aqueous conditions the thioester compounds can then be reacted with the N-terminal cysteine-containing EPO with the end product consisting of EPO conjugated to said thioester compound via an amide bond formed between the a-amino group of the cysteine residue and carboxyl carbon of said thioester (Tam, J. P., Proc. Natl. Acad. Sci. 92, 12485-12489 (1995)).

Examples of derivatized erythropoietic compounds of the invention are:

M-PEG-A-Cys-EPO where Cys represents Cys₋₁ relative to the bioactive erythropoietin amino acid sequence; M is a lipid, carbohydrate, polysaccharide, fatty acid, fatty acid derivative, fatty alcohol or protein; and A represents the carrier or reaction product of the electrophilic thiol reactive group, preferably maleimide.

(M-PEG)₂-A-Cys-EPO where Cys represents Cys₋₁ relative to the bioactive erythropoietin amino acid sequence; where the M-PEG is esterified to two different carboxyl groups on A and A further comprises a moiety that represents the carrier or reaction product of the electrophilic thiol reactive group, preferably maleimide; and where M is a lipid, carbohydrate, polysaccharide, fatty acid, fatty acid derivative, fatty alcohol or protein. Higher multiples are included as well.

(M-PEG)₂-R-A-Cys-EPO where Cys represents Cys₋₁ relative to the bioactive erythropoietin amino acid sequence; where A represents the carrier or reaction product of the electrophilic thiol reactive group, preferably maleimide; where the (M-PEG)₂-R is attached to two different carboxyl groups on A; where M is a lipid, carbohydrate, polysaccharide, fatty acid, fatty acid derivative, fatty alcohol or protein and R is a valency enhancing construct. Higher multiples are included as well.

M-A-Cys-EPO where Cys represents Cys₋₁ relative to the bioactive erythropoietin amino acid sequence; where M is a protein or peptide and A is a free cysteine side chain on said protein or peptide.

M-A-Cys-EPO where Cys represents Cys₋₁ relative to the bioactive erythropoietin amino acid sequence and where M is a lipid and A where A represents the carrier or reaction product of the electrophilic thiol reactive group, preferably maleimide.

M-A-Cys-EPO where Cys represents Cys₋₁ relative to the bioactive erythropoietin amino acid sequence; where M is biotin, dansyl, or other moiety imparting biophysical characteristics to EPO that are useful for research, diagnostic or therapeutic purposes; and where A represents the carrier or reaction product of the electrophilic thiol reactive group, preferably maleimide. In the case where biotin or another moiety having a known binding partner is incorporated into the conjugate, it is anticipated that said conjugate may be used in research, diagnosis or therapy in a complex with its known binding partner such as in a biotin-avidin complex.

M-A-Cys-EPO where Cys represents Cys₋₁ relative to the bioactive erythropoietin amino acid sequence; where M is a protein, peptide, or other moiety imparting unique characteristics to EPO that are useful for research, diagnostic or therapeutic purposes; and where A represents the product of the reaction between an ester or thioester group and Cys₋₁.

Therapeutic Adminsitration

The NTFR erythropoietin conjugates of the present invention are useful as a parenteral formulation in treating blood disorders characterized by low or defective red blood cell production such as various forms of anemia, including anemia associated with chronic renal failure, zidovidine treated HIV infected patients, and cancer patients on chemotherapy. It may also have application in the treatment of a variety of disease states, disorders and states of hematologic irregularity such as sickle cell disease, beta-thalassemia, cystic fibrosis, pregnancy and menstrual disorders, early anemia of prematurity, spinal cord injury, space flight, acute blood loss, aging and the like. It may also have application in situations where an increase in red blood cells is desired such as in pre-surgery patients. Preferably, the NTFT erythropoietin conjugate composition of the present invention is administered parenterally (e.g. IV, IM, SC or IP).

Effective dosages are expected to vary considerably depending on the condition being treated and the route of administration but are expected to be in the range of 0.1 to 100 μg/kg body weight (approximately 7 to 7000 U) of the active material. Preferable doses for treatment of anemic conditions are about 50 to about 300 Units/kg three times a week.

The NTFT erythropoietin conjugate formulations of the present invention are useful in treating neurological pathologies particularly those of the central nervous system, including, but not limited to: cerebral and spinal ischemia, acute brain injury, spinal cord injury, retinal disease, and neurodegenerative diseases such as Alzheimer's disease, Parkinson's disease, Huntington's disease, and ALS. In addition, to neurological pathologies, the EPO formulations of the present invention are useful in treating disorders or enhancing healing of other tissue damaged as a result of ischemic or hypoxic stress such as the infracted heart, soft tissue damage as a consequence of surgical intervention including connective tissue and organ damage, as well as tissue damage as a result of trauma or immune-mediated inflammation.

Pharmaceutical Compositions

The therapeutic NTFT protein conjugates prepared in accordance with this invention may be prepared in pharmaceutical compositions suitable for injection with a pharmaceutically acceptable carrier or vehicle by methods known in the art. For example, appropriate compositions have been described in WO97/09996, WO97/40850, WO98/58660, and WO99/07401. Among the preferred pharmaceutically acceptable carriers for formulating the products of the invention are human serum albumin, human plasma proteins, etc. The compounds of the present invention may be formulated in 10 mM sodium/potassium phosphate buffer at pH 7 containing a tonicity agent, e.g. 132 mM sodium chloride. Optionally the pharmaceutical composition may contain a preservative. The pharmaceutical composition may contain different amounts of erythropoietin products, e.g. 10-2000 μg/ml, e.g. 50 μg or 400 μg.

The stability of the composition can be further enhanced by the addition of antioxidants such as tocopherol, butylated hydroxytoluene, butylated hydroxyanisole, ascorbyl palmitate, or edetates such as e.g. disodium edetate, with the edetates additionally binding possibly present heavy metals. The stability can furthermore be enhanced by the addition of preserving agents such as benzoic acid and parabens, e.g. methylparaben, and/or propylparabene.

Treating Blood Disorders Characterized by Low or Defective Red Blood Cell Production

In one aspect of the invention, the administration of the NTFT erythropoietin conjugates of the present invention is directed to causing increased red cell formation in humans. Therefore, administration of the NTFT erythropoietin conjugates replenishes or substitutes for the function of the naturally occurring EPO protein that is important in the production of red blood cells. The pharmaceutical compositions containing the NTFT erythropoietin conjugates may be formulated at a strength effective for administration by various means to a human patient experiencing a blood disorders characterized by low or defective red blood cell production, either alone or as part of a condition or disease. The pharmaceutical compositions may be administered by injection such as by subcutaneous, intravenous or intramuscular injection. Average quantities of the NTFT erythropoietin conjugates may vary and in particular should be based upon the recommendations and prescription of a qualified physician. The exact amount of conjugate is a matter of preference subject to such factors as the exact type of condition being treated, the condition of the patient being treated, as well as the other ingredients in the composition. For example, 0.01 to 10 μg per kg body weight, preferably 0.1 to 10 μg per kg body weight, may be administered e.g. once weekly.

In another aspect of the invention, the use of the NTFT erythropoietin conjugate formulations of the invention is directed to treating human patients in need of intervention to protect, restore, or enhance neurological tissues, particularly those of the central nervous system, and functions diminished, compromised, or lost due to: cerebral and spinal ischemia, acute brain injury, spinal cord injury, retinal disease, and neurodegenerative diseases such as Alzheimer's disease, Parkinson's disease, Huntington's disease, and ALS. The use of the NTFT erythropoietin conjugate formulations of the present invention can also be directed to treating human patients in need of intervention to protect, restore, or enhance healing of other tissue damaged as a result of ischemic or hypoxic stress such as the infracted heart, soft tissue damage as a consequence of surgical intervention including connective tissue and organ damage, as well as tissue damage as a result of trauma or immune-mediated inflammation.

Throughout this application, various publications have been referenced. The disclosures in these publications are incorporated herein by reference in order to describe more fully the state of the art.

The present invention is further illustrated by the following examples that are presented for purposes of demonstrating, but not limiting, the preparation of the compounds and compositions of this invention.

### EXAMPLE 1

### Cloning Cys-EPO

The N-terminus of EPO is not involved in receptor binding and is positioned such that it points away from the EPO-receptor complex. Because of this, the N-terminus of EPO offers an ideal position for incorporating chemical modifications that should have the least steric effect on receptor binding and therefore also on bioactivity. Introduction of a cysteine residue at the N-terminus would therefore allow for site-specific modification of EPO without disrupting receptor binding.

The creation of a HEPO sequence that has a cysteine residue N-terminal of the alanine residue by manipulating the EPO genetic sequence or cDNA was therefore undertaken. However, in silico analysis, suggests that merely adding a cysteine codon into the EPO precursor coding sequence could shift the putative cleavage site upstream between proline24 and valine25 in the signal peptide to leave val25 as the neo-N-terminal residue or cause the cleavage between the cys(−1) and ala1 to effectively remove the added cysteine entirely (SignalP 3.0; www.cbs.dtu.dk/services/SignalP/). To increase the probability that the N-terminus of an engineered EPO would be cysteine, it was proposed to replace the endogenous HEPO signal peptide with one that is known to be efficient at targeting proteins to the ER e.g. the first 26 amino acids of human growth hormone (Morris, A. E. et al. (1999) Journal of Biological Chemistry 274, 418-423). When analyzed by computer models, this heterologous protein (FIG. 1, SEQ. ID. NO: 3)was predicted to yield a mature protein with an N-terminal cysteine.

To accomplish the production of a construct that can be used to express human EPO with an additonal cysteine at the N-terminus, the human growth hormone leader sequence was engineered, along with the cysteine codon into a vector for expression of the novel protein.

The nucleic acid sequence of EPO was amplified from pEG15. The nucleic acid sequence for the hGH signal sequence was amplified from a vector which originated from pXGH5 (Nichols Diagnostic). The hGH-EPO construct was ligated into a plasmid designated pSUE plasmid. The method used to create a polynucleotide coding for the polypeptide of SEQ ID NO: 3 (FIG. 1) is described below.

Primer Design

The first PCR Primer Pair (SEQ ID NOS: 15 and 16) was used to generate a 107 bp fragment containing HindIII-Kozac-hGH-CYS-ApaLI.

5′.HindIII.Kozac.hGH
5′-ATG CAA GCT TGC CAC CAT GGC TAC AG-3′
3′.hGH.cys.ApaLIB
5′-GTG GTG GTG CAC AGG CAC TGC CCT C-3′

A second PCR Primer Pair (SEQ ID NOS: 17 and 18) was used to generate a 518 bp fragment containing CYS-ApaLI-EPO-BamHI.

5′.cys.ApaLI.EPO
5′-ATG CGC ATG TGC ACC ACC ACG CCT C-3′
3′EPO.BamHI
5′-GCA TGG ATC CTC TGT CCC CTG-3′

Cloning

The first primer pair was used to generate the hGH signal sequence fragment (SEQ ID NO: 2) for the final construct. PCR gradient conditions were 95° C.×2 min followed by 30 cycles of 95° C.×2 min, 50° C. to 60° C. for 30 sec, 72° C. for 30 sec, followed by a final extension at 72° C. for 3 minutes and then a 4° C. hold. The second primer pair was used to generate the EPO fragment of the final construct. PCR conditions were 94° C. for 2 min then 30 cycles of 94° C.×30 sec, 60° C.×30 sec, 72° C.×3 min, ending with a final extension at 72° C. for 7 min and a 4° C. hold. After amplification, 10 uL of each PCR reaction was combined with 1 uL of 10× loading dye and run on a 1% SeaKem gel (Bio-Rad, Hercules, Calif.) with 1 Kb ladder (Invitrogen, Carlsbad, Calif.). The hGH PCR reaction generated a band migrating at about the 100 bp position at each temperature; the bands were excised and pooled together and extracted according to instructions for the QIAQuick Gel Extraction Column (Qiagen, Valencia, Calif.) and eluted in 30 uL dH₂O. The EPO band migrating at the around 500 bp position was similarly excised and extracted.

The EPO fragment was digested with BamHI and ApaLI, the hGH fragment was digested with HindIII and ApaLI, and the vector pSUE was digested with HindIII and BamHI. Ten uL of pSUE digest was run on a 1% SeaKem gel as described above. The vector band at ˜10 Kb was excised and extracted as described above. The entire 30 uL eluate was treated with calf intestinal alkaline phosphatase (New England BioLabs, Beverly, Mass.), then purified according to the instructions for QIAQuick PCR Purification column (Qiagen) and eluted in 30 uL dH₂O. The fragment digests were purified with the QIAQuick PCR Purification column and eluted in 30 uL dH₂O.

Ligation of the individual fragments and the vector was performed using Roche Rapid Ligation Kit (Roche Applied Science, Indianapolis, Ind.). The ligation reaction was transformed into TOP10 OneShot chemically competent cells (Invitrogen) and plated on Luria-Bertani (LB) plates containing 100 ug/mL ampicillin (Teknova, Half Moon Bay, Calif.). Individual colonies were picked into selective liquid LB media and grown overnight at 37 degrees shaking at 225 rpm. Plasmid DNA was extracted using Qiagen Spin Miniprep Kit (Qiagen) and eluted into 75 uL dH₂O. All clones were digested restriction enzyme to screen for insert. Positive clones were sequenced using fluorescent dye-terminators and the ABI3100 Genetic Analyzer (Applied Biosystems, Foster City, Calif.) with primers internal to the vector. Two positive clones were identified by sequencing; however, both contain a mutation in the hGH signal sequence (Q22R). This mutation does not affect predicted cleavage at C-terminal cysteine. The final plasmid was termed pSUEcysEPO.

In this work we have used a simple expression vector utilizing a strong viral promoter, a consensus Kozak sequence, the gene of interest (EPO), a hexaHis tag, a stop codon and a polyadenylation signal derived from the bovine growth hormone gene. Alternatively, a stable mammalian cell line expressing cys-EPO could have been generated to express the gene product. HEK 293E cells were used, however, any host cell (preferred mammalian, but not obligatory), capable of processing mammalian signal sequences could have been used.

### EXAMPLE 2

### Expression of Cys-EPO

The novel EPO protein was expressed using transient transfection where DNA is taken-up by mammalian cells, exported to the nucleus and transcribed. Using this technique a pulse of protein expression achieved in a rapid fashion. The product, cys-EPO was collected from the conditioned medium five days after transfection and purified using the hexaHis tag positioned at the C-terminus of the protein.

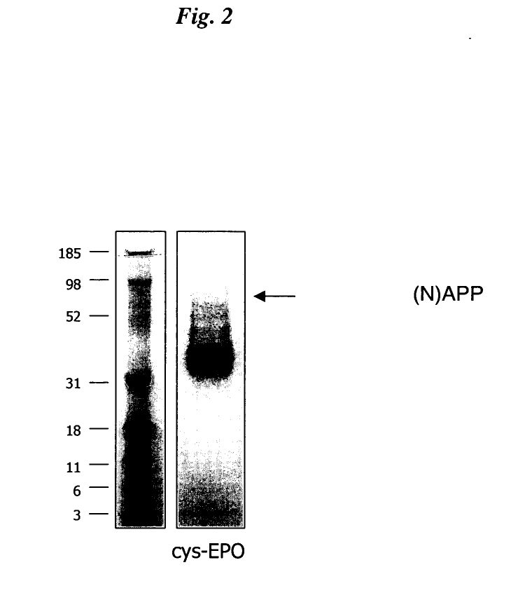

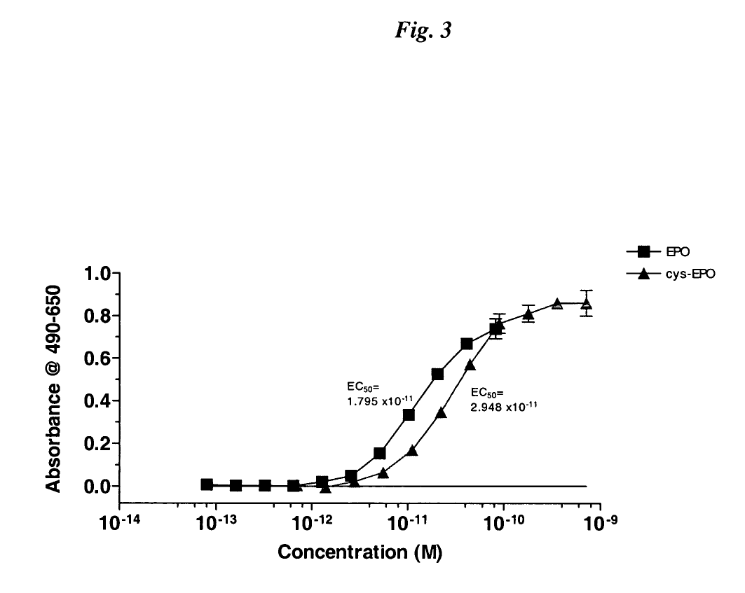

DNA encoding cys-EPO (pSUEcysEPO) was transfected into HEK 293E cells using a cationic lipid reagent (LF2K). Cells were then cultured in a serum-free medium (293-SFMII) in a 10-tier cell factory and after 4 days conditioned medium was recovered and cys-EPO was purified using TALON IMAC. Following dialysis and concentration, the purified product was analyzed by SDS PAGE for purity (FIG. 2), N-terminal sequencing and UT-7 bioassay (FIG. 3).

In the bioassay, UT-7 cells starved in IMDM with L-glu and 5% FBS without Epo for 24.5 hrs prior to assay. Cells were washed and plated at 30,000 cells per well. EPO (2.5-0.0024 ng/mL) and cys-EPO (20-0.01952 ng/mL) were added in duplicate. After 47.2 hrs at 37C and the cell number per well was measured using Promega's MTS solution with OD readings taken at 1, 2 and 3 hr intervals. Values were background corrected with SoftMax Pro and the average background was 0.327 OD units (FIG. 3).

The fact that the cys-EPO was recovered from the supernatent confirmed the successful expression of the protein from the nucleic acid transfected into the cells and indicated that the protein was targeted to the ER by the human growth hormone signal sequence, folded correctly and secreted.

The Coomasie stained SDS PAGE (FIG. 2) of the material shows a dominant band at about 31 kDa which matches well the 34 kDa attributed to the natural glycosylated product produced in humans and by mammalian cells. In contrast, the nonglycosylated EPO is about 18 kDa

N-terminal sequencing confirmed the existence of a single amino acid upstream of the normal mature alaninel residue. The N-terminal sequence for the material removed from this band are indicated and the * is explained as follows: Since cysteine residues are not easily recognized by the N-terminal sequencing method, unless the protein is derivatized in some manner, the N-terminal amino acid (N in parenthesis) can only be designated as present i.e. ‘called’ by the sequencer, and therefore indicates that the protein does not have a blocked N-terminus. In addition, it can be determined that (N) is not an amino acid derived from the C-terminus of the hGH signal sequence as this is an alanine (A) which can be identified by the sequencing method. Futhermore, the series of amino acids after the first call (N) correspond to the mature human EPO gene (APP etc.).

The nucleic acid sequence coding for the construct is given in SEQ ID NO: 19.

### EXAMPLE 3

### Chemical Modifications of Cys-EPO

Experiment 1

Buffer exchange was performed on Cys-EPO against phosphate buffered saline at pH 7.0 (PBS) with 1 mM ethylenediaminetetraacetic acid (EDTA). Cys-EPO (0.7 mg/ml in PBS+100 mM phosphate, pH 6.8) and EPO (0.7 mg/ml in PBS+100 mM phosphate, pH 6.8) were incubated at 37° C. for 2 hours with 0 mM, 15 mM, 20 mM, and 25 mM b-mercaptoethylamine (MEA) (Pierce Biotechnology, Inc., Rockford, Ill.). The MEA was then removed from the samples with Biospin-6 desalting columns (Biorad Laboratories, Hercules, Calif.) equilibrated with phosphate buffer (50 mM, pH 6.8) as per manufacturers instructions. The samples were then incubated with 0.75 mM maleimide-PEG (average molecular weight: 5960) (Nektar, Huntsville, Ala.) for 1 hour at ambient temperature. After an hour, cysteine was added to a concentration of 0.75 mM and incubated at ambient temperature for 20 minutes. Samples were then loaded and run on a 4-12% SDS-PAGE gel. Samples were also analyzed by SELDI mass spectrometry (Ciphergen, Fulton, Calif.) on H4 reversed-phase chips using a matrix of sinnapic acid and prepared as per manufacturers recommendations. Wild type EPO was treated in an identical manner.

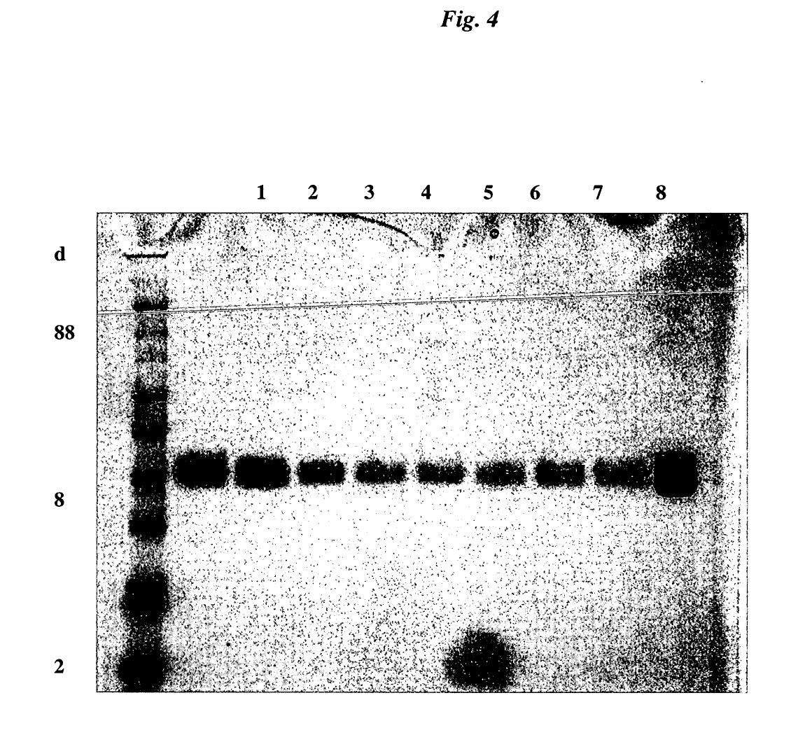

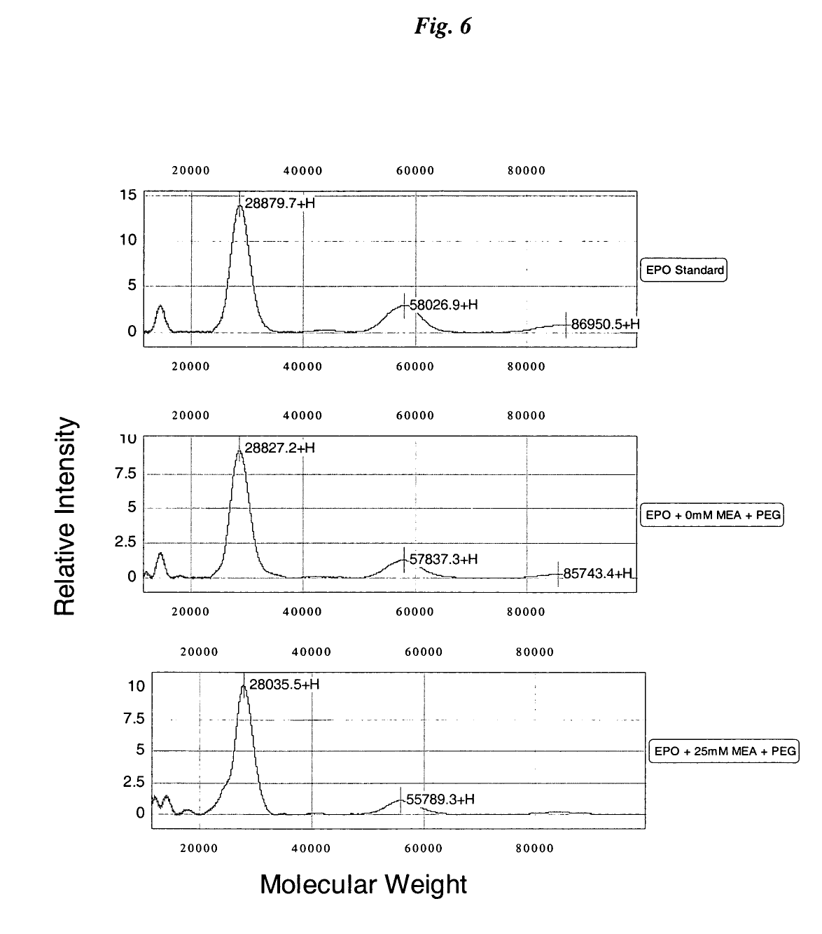

The gel of EPO samples (FIG. 4) shows that no appreciable PEGylation of EPO occurred under any of the conditions studied. This is indicated by the lack of any bands representing a molecular weight shift relative to the unmodified EPO standard. The SELDI-MS of the samples (FIG. 6) also shows no molecular weight difference, indicating again that no PEGylation occurred under these conditions.

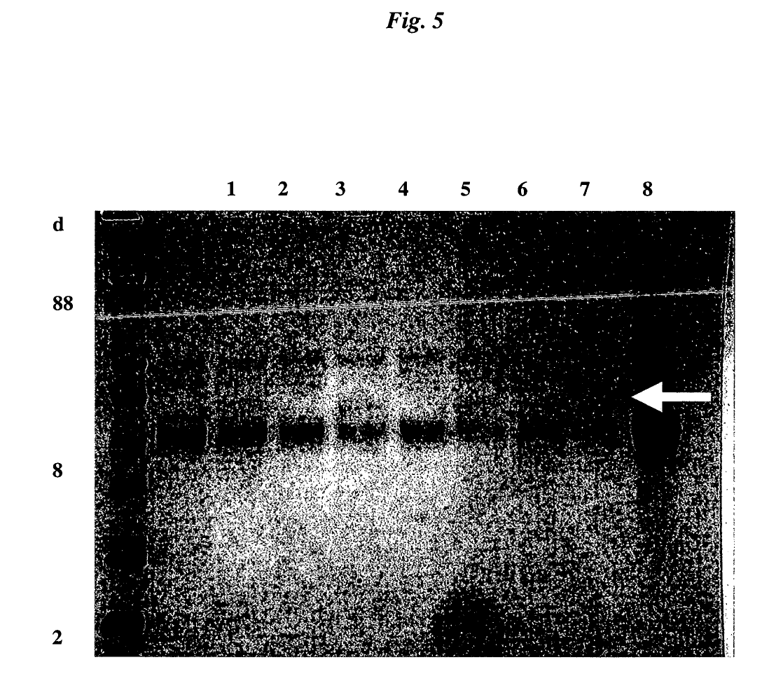

The gel of cys-EPO samples (FIG. 5) shows that appreciable PEGylation of cys-EPO occurred under each of the conditions studied. This is indicated by the bands (indicated by the white arrow) representing a molecular weight shift relative to the unmodified cys-EPO standard (Note that the cys-EPO standard was loaded at a higher concentration to better show the presence of impurities and that the molecular weight shift described is in relation to the main band observed for the standard).

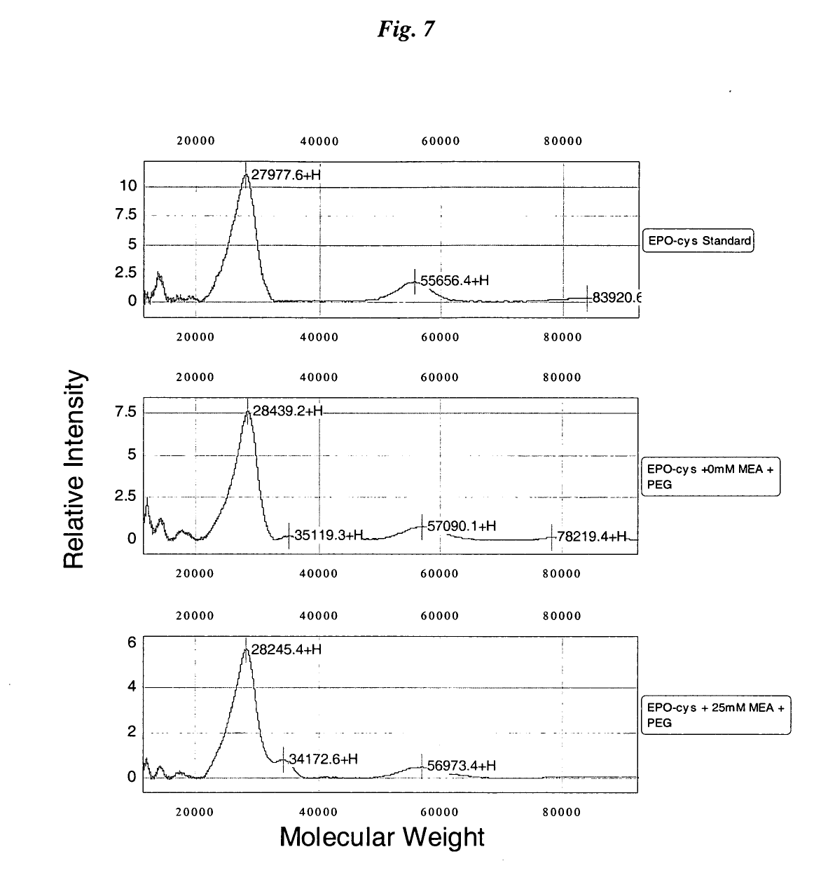

In the SELDI mass spectral analysis of the samples (FIG. 6 and 7), the peaks around 28,000 correspond to unmodified EPO or cys-EPO. Note the presence of peaks corresponding to the addition of a 5,960 MW PEG in FIG. 7, a molecular weight difference proportional to that expected for the PEGylated product. Due to the heterogeneity of the PEG and the glycosylation on EPO, the peaks are quite broad and the molecular weights must be viewed as approximate. However, the relative molecular weights indicate the attachment of PEG to both unreduced and reduced Cys-EPO, indicating again that PEGylation of the cys-EPO does occur under these conditions. Also, the degree of PEGylation appears to increase relative to the concentration of MEA used for reduction. This indicates that at least some of the N-terminal cysteine on cys-EPO is disulfide bridged to another thiol such as cysteine or glutathione and that this disulfide can be selectively reduced using the conditions described here. Taken together, these data show that the N-terminal thiol on cys-EPO is accessible and can be modified by a thiol specific reagent such as maleimide-PEG.

Experiment 2

Buffer exchange is performed on Cys-EPO against phosphate buffered saline at pH 7.0 (PBS) with 1 mM ethylenediaminetetraacetic acid (EDTA). To this solution is added a 3-fold molar excess of maleimide-activated disteroylphosphatidylethanolamine containing a polymer linker (such as PEG) of molecular weight 14 to 20,000 between the maleimide and lipid (mal-PEG-DSPE). The reaction mixture is incubated between 20 and 25 degrees Celsius for one hour. The reaction mixture is then loaded onto a zorbax GF-250 XL size exclusion HPLC column and eluted with PBS at a flow rate of 2 ml/minute. The fractions containing the resulting modified protein peak are then pooled and tested for bioactivity.

Experiment 3

To 28.7 ml of 0.5 M HOBt/HBTU (1-hydroxybenzotriazole/2-(1H-benzotriazol-1-yl)-1,1,3,3-tetramethyluronium hexafluorophosphate) in dimethylformamide (DMF) is added 14.35 ml of 2 M diisopropylethylamine (DIEA) followed by 5 g (14.35 mmol) of 3-(S,trityl)-mercaptopropionic acid (Bachem, King of Prussia, Pa.). The solution is added to 5 g of MEHA resin (0.8 mmol/g) (Bachem) and agitated for 30 min. The resin is washed with several volumes of DMF, DCM and methanol in succession and dried in vacuo. The resulting resin is used for peptide synthesis with standard Boc chemistry. Peptides of 2 to 50 residues in length are synthesized using standard Boc chemistry and cleavages are accomplished in 90% HF, 10% p-cresol, −5° C., 1.5 h. Peptides are precipitated in ether, lyophilized, and purified by preparative reversed-phase HPLC (RP-HPLC). Peptides are then incubated with Cys-EPO in PBS (pH 7.0)+1 mM EDTA for 12 to 48 hours. Reactions are monitored with analytical size-exclusion chromatography HPLC (SEC-HPLC), SDS gel electrophoresis and/or SELDI mass spectrometry. Final conjugates are then loaded onto a Zorbax GF-250 XL size exclusion HPLC column and eluted with PBS at a flow rate of 2 ml/minute. The fractions containing the resulting modified protein peak are then pooled and tested for bioactivity.

### EXAMPLE 4

### UT7 Cell Proliferation Assay

UT7 is a human leukemic cell line that has been adapted to become EPO dependant (Komatsu, N., et al. Blood 82(2), 456-464, 1993). The UT7 cells are washed three times in PBS and starved for EPO for 24 hours prior to assay. UT-7 cells were starved in IMDM media with added L-glutamine and FBS at 5% (15Q). Cells are washed once in 50 mL DPBS and counted while suspended in DPBS and suspended in the appropriate media to a final concentration of 6×10⁵ cells/mL (yields a final concentration of 30,000 cells per well). An EPO standard is prepared by diluting EPO stock (1.7 mg/mL) to 0.85 μg/mL (2 μL in 4 mL media). The stock solution is diluted 2:340 to 5 ng/mL followed by 1:2 serial dilutions down to a concentration of 0.0098 ng/mL in I5Q media. The resulting dilutions provides standards at concentrations of 2.5 ng/mL to 0.0024 ng/mL. The test sample is diluted in a similar manner. A 50 μL aliquot of the UT-7 cell suspension is transferred to the corresponding wells and the plates were incubated at 37° C. for 48 hours. Cell proliferation is assessed using Promega's MTS solution (Promega, Madison, Wis.), adding 20 μL per well. Readings begin 1 hour after MTS addition.

FIG. 3 shows a graph of the concentration dependence of the EPO material in UT7 cell assay performed on unmodified and N-terminal cys-modified EPO. The EC₅₀ calculated from the data for unmodified EPO is 1.795×10⁻¹¹ M and for modified EPO 2.948×10⁻¹¹ M. These data indicate the secreted material is active.

### EXAMPLE 5

### Stimulation of the Hematopoiesis in Mice

BDF1 female mice obtained from Charles Rivers Laboratories (Raleigh, N.C.), weighing approximately 18-20 grams are group housed (10 per cage) in filtered-top plastic cages.

On Day -5 of the study, the animals are assigned to 1 of 3 treatment groups (PBS control, EPO and M-PEG-A-Cys-EPO) with 15 animals in each group. The animals are anesthetized with CO₂ and blood samples taken in EDTA coated glass tube via retro-orbital sinus with a target blood volume of 0.05 mL/sample to establish baseline levels. Blood is placed into commercially available EDTA prepared microcentrifuge tubes. Aliquots are placed into hematocrit tubes and the tubes are sealed with clay and centrifuged for 5 minutes. The Packed Cell Volume (PCV/hematocrit) is obtained from reading the hematocrit tubes on a commercially available hematocrit determinator card. Using 10 μl of blood, hemoglobin levels are determined using a Coulter™ Counter. On Days 0 and 2, the animals receive an intraperitoneal injection of 0.94 mL (112.8 mL/kg) of either PBS (pH 7.4), EPO (0.333 μg/mL in PBS), or an M-PEG-A-Cys-EPO composition of the invention in an amount which is equivalent in bioactivity as calibrated by the UT-7 assay of Example 4) in PBS. On days 4, 7,10, 14, 17, and 21 blood samples are taken and aliquoted into hematocrit tubes sealed with clay and centrifuged for 5 minutes. The packed cell volume (PCV/hematocrit) is obtained by reading the hematocrit tubes on a commercially available hematocrit determinator card. Hemoglobin levels are determined using a Coulter™ Counter using 10 μl samples.

1. A method of preparing a therapeutic protein conjugate having a polymer conjugated to an N-terminal cysteine of the therapeutic protein wherein the thiol of said cysteine residue participates in the formation of a covalent bond of said conjugate comprising:
a) obtaining a nucleic acid sequence for said therapeutic protein,
b) choosing a signal sequence for expression of said protein in a cell and obtaining a nucleic acid sequence for said signal sequence,
c) directing the formation of a construct by the engineering of the signal sequence of (b) to the protein sequence of (a) with the codon TGT interposed between them so that the signal sequence is upstream of the TGT,
d) causing said construct to be expressed in the cell,
e) recovering the polypeptide coded for by said construct, and
f) conjugating said polypeptide at the N-terminal cysteine to a polymer.

2. A method of claim 1 where the directing step is oligonucleotide-directed mutagenesis.

3. A method of claim 1 where the choosing step involves the use of a publicly available computer method.

4. The method of claim 3 wherein the possible signal sequences are selected from the group consisting of the human growth hormone leader (SEQ ID NO: 2), an antibody heavy chain leader sequence (SEQ ID NO: 3), an antibody light chain leader sequence (SEQ ID NO: 4), a human interferon delta1 leader sequence (SEQ ID NO: 12), and a human interferon omega I sequence (SEQ ID NO: 13).

5. An erythropoietic conjugate having the biological properties of causing bone marrow cells to increase production of red blood cells, comprising a moiety of the formula
(M)n-X-A-cys-EPO (I)
where EPO is an erythropoeitin moiety selected from erythropoietin or an erythropoietin variant having at least one amino acid different from the wild-type human EPO, or any pharmaceutical acceptable derivatives thereof having biological properties of causing bone marrow cells to increase production of red blood cells, cys represents the amino acid cysteine and occurs at position −1 relative to the amino acid sequence of the erythropoietin moiety; A is a residue of a thiol reactive moiety; X is a hydrophilic polymer and is optional; M is an organic molecule capable of increasing the circulating half-life of the moiety and n is an integer from 0 to 15.

6. The erythropoietic conjugate of claim 3 that causes bone marrow cells to increase production of red blood cells, and said increase is sustained after administration of said erythropoietin conjugate for a greater period of time than that seen after administration of unconjugated erythropoietin.

7. The erythropoietic conjugate of claim 4, where the sustained effect is due to increased serum half life over unmodified mammalian erythropoietin.

8. The erythropoietic conjugate of claim 3 wherein the moiety M comprises one to about six organic moieties, which are each independently selected from a fatty acid group, a fatty acid ester group, a lipid or a phospholipid.

9. The erythropoietin conjugate of claim 4 wherein the hydrophilic polymer is a polyalkylene oxide.

10. The erythropoietic conjugate of claim 3, wherein said erythropoietin or erythropoietin moiety is selected from recombinant and non-recombinant mammalian erythropoietin.

11. The erythropoietic conjugate of claim 7, wherein the polyalkylene oxide is a substituted polyethylene oxide.

12. The erythropoietic conjugate of claim 7, wherein the polyalkylene oxide is selected from polyethylene glycol homopolymers, polypropylene glycol homopolymers, alkyl-polyethylene oxides, bispolyethylene oxides and co-polymers or block co-polymers of polyalkyene oxides.

13. The erythropoietic conjugate of claim 7, wherein said polyalkylene oxide is a polyethylene glycol homopolymer having a molecular weight of between about 200 and about 100,000.

14. The erythropoietic conjugate of claim 3 wherein said hydrophilic polymer is a linear or branched polyalkane glycol chain, a carbohydrate chain, an amino acid chain or a polyvinyl pyrolidone chain, and wherein said hydrophilic polymer has a molecular weight of about 800 to about 120,000 Daltons.

15. The erythropoietic conjugate of claim 12 wherein said hydrophilic polymer is a linear or branched polyalkane glycol chain with a molecular weight greater than 2,000 Daltons.

16. The erythropoietic conjugate of claim 12 wherein said hydrophilic polymer is a linear or branched polyethylene glycol chain or a linear or branched substituted polyethylene glycol chain and the organic moiety M is selected from an alkyl group, a C₆-C₄₀ fatty acid group, a C₆-C₄₀ fatty acid ester group, a lipid group and a phospholipid group.

17. The erythropoietic conjugate of claim 14 wherein said hydrophilic polymer is a linear or branched polyethylene glycol chain that is terminally substituted with an organic moiety selected from an allkyl group, a C₆-C₄₀ fatty acid group, a C₆-C₄₀ fatty acid ester group, a lipid group or a phospholipid group.

18. The erythropoietic conjugate of claim 15 wherein said organic moiety is palmitoyl.

19. The erythropoietic conjugate of claim 15 wherein the organic moiety is disteroylphosphatidyl ethanolamine (DSPE).

20. The conjugate of claim 3 where A is ethyl, X is PEG or other polymer and is optional, and M is biotin, dansyl, or other moiety imparting biophysical characteristics to EPO that are useful for research, diagnostic or therapeutic purposes.

21. The conjugate of claim 3 where A is ethyl.

22. An erythropoietic conjugate of claim 3 where EPO is an erythropoietin moiety selected from the group consisting of a) SEQ ID NO: 1 from position 28 to at least position 165, b) an erythropoietin variant having at least one amino acid different from the SEQ ID NO: 1, or c) any pharmaceutical acceptable derivatives of (a) or (b); and cys represents the amino acid cysteine and occurs at the N-terminal position relative to amino acid number 28 of SEQ ID NO: 1 or variant; A indicates the residue of a thiol reactive moiety; X is a hydrophilic polymer; and M is an allkyl group, a C₆-C₄₀ fatty acid group, a C₆-C₄₀ fatty acid ester group, a lipid group or a phospholipid group; and n is an integer from 0 to 15.

23. A method of preparing an erythropoietic conjugate of claim 3 comprising contacting a cys-EPO moiety having a cysteine residue at the N-terminus with a preconstructed hydrophilic polymer—organic moiety complex of the formula Y—X-(M)ₙ, where Y is a thiol reactive moiety which thiol reactive moiety contains or becomes the residue A under conditions such that an EPO-cys-polymer-conjugate is formed.

24. The method of claim 21, wherein said polymer is a polyalkylene oxide.

25. The method of claim 22, wherein said polyalkylene oxide is an alpha-substituted polyalkylene oxide.

26. The method of claim 23, wherein said polyalkylene oxide is a polyethylene glycol.

27. The method of claim 21, wherein the thiol reactive moiety is a sulfone.

28. The method of claim 25, wherein the thiol reactive moiety is a ethyl sulfone.

29. The method of claim 21, wherein the thiol reactive moiety is a disulfide .

30. The method of claim 21, wherein the thiol reactive moiety is a maleimide.

31. The method of claim 21, wherein X is a peptide or protein and A is the reaction product of Cys₋₁ and a thioester or ester moiety.

32. The method of claim 21, wherein the thiol reactive moiety is a iodoacetamide.

33. The method of claim 21 where A is ethyl, X is PEG or other water soluble polymer and is optional, and M is biotin, dansyl, or other moiety imparting biophysical characteristics to EPO that are useful for research, diagnostic or therapeutic purposes.

34. A method of treating anemia comprising administering a therapeutically effective amount of conjugate of claim 3.

35. The method of claim 32 wherein said conjugate is characterized by increased serum half-life-compared to the unconjugated erythropoietin.

36. An erythropoietic protein or protein conjugate containing recombinant or non-recombinant mammalian erythropoietin in which a cysteine residue having a free alpha amine has been added, by recombinant, enzymatic or chemical means to provide a reactive free thiol and which reactive free thiol does not interfere with protein folding, secretion, or bioactivity, and which thiol may be derivatized thereby increasing the circulation half life or otherwise improving the biological activity of said erythropoietic protein.

35. A moiety of the formula: Z-cys-EPO; where EPO is an erythropoeitin moiety selected from erythropoietin or an erythropoietin variant having at least one amino acid different from the wild-type human EPO, or any pharmaceutical acceptable derivatives thereof having biological properties of causing bone marrow cells to increase production of red blood cells, cys represents the amino acid cysteine and occurs at position −1 relative to the amino acid sequence of the erythropoietin moiety; and Z is a heterologous signal sequence.

36. A moiety of claim 35 wherein the heterologous signal sequence is the human growth hormone leader sequence (SEQ. ID No. 2).
